(19) United States
(12) Patent Application Publication
(10) Pub. No.: US 2023/0372691 A1
(43) Pub. Date: Nov. 23, 2023

(54) CONVERSION ADAPTER, CONNECTOR SET, AND ASSEMBLY

(71) Applicant: TERUMO KABUSHIKI KAISHA, Tokyo (JP)

(72) Inventors: Tasuku Kuriyama, Kai-Shi (JP); Yasuhiro Ueda, Kofu-Shi (JP)

(73) Assignee: TERUMO KABUSHIKI KAISHA, Tokyo (JP)

(21) Appl. No.: 18/363,355

(22) Filed: Aug. 1, 2023

Related U.S. Application Data
(63) Continuation of application No. PCT/JP2021/046119, filed on Dec. 14, 2021.

(30) Foreign Application Priority Data
Feb. 24, 2021 (JP) 2021-027894

(51) Int. Cl. A61M 39/10

(52) U.S. Cl. CPC A61M 39/1011

(57) ABSTRACT

A conversion adapter is attachable to a female connector part comprising a male screw part to which a luer lock-type first male connector part conforming to ISO 80369 is connectable, and comprises: a female screw part screwable to the male screw part of the female connector part; and a locking unit configured to lock to a second male connector part not conforming to ISO 80369.

### CROSS-REFERENCE TO RELATED APPLICATIONS

This is a bypass application of PCT Application No. PCT/JP2021/046119, filed on Dec. 14, 2021, which claims priority to Japanese Application No. JP2021-027894, filed on Feb. 24, 2021. The contents of these applications are hereby incorporated by reference in their entireties.

### BACKGROUND

The present disclosure relates to a conversion adapter, a connector set, and an assembly.

Conventionally, a medical male connector part conforming to ISO 80369, which is an international standard of the International Organization for Standardization, has been known. In contrast, for example, a male connector part not conforming to ISO 80369 might be used in a medical connector and the like used for a specific application.

JP 2013-192602 A discloses an adapter detachably attached to a mixed injection port. By attaching the adapter disclosed in JP 2013-192602 A to the mixed injection port, both the lock-type luer part and slip-type luer part may be connected to the mixed injection port.

### SUMMARY

From the viewpoint of work efficiency of a medical worker in a medical site, it is desirable that a female connector part connectable to a luer lock-type male connector part conforming to ISO 80369 may be easily converted into a female connector part connectable to a male connector part not conforming to ISO 80369 in a locked state using a predetermined lock mechanism.

An object of the present disclosure is to provide a conversion adapter capable of easily converting a female connector part connectable to a luer lock-type male connector part conforming to ISO 80369 into a female connector part connectable to a male connector part not conforming to ISO 80369 in a locked state using a predetermined lock mechanism, a connector set including the conversion adapter, and an assembly provided with the conversion adapter.

According to a first aspect of the present disclosure, a conversion adapter is attachable to a female connector part on which a male screw part to which a luer lock-type first male connector part conforming to ISO 80369 is connectable is formed, and comprises a female screw part screwable to the male screw part of the female connector part, and a locking unit capable of locking a second male connector part not conforming to ISO 80369.

According to one embodiment, the conversion adapter comprises a cylindrical adapter body, in which the female screw part is formed on an inner surface of the adapter body, and the locking unit is formed on an outer surface of the adapter body.

According to one embodiment, the adapter body comprises an inner cylindrical body provided with an inner cylindrical part on an inner surface of which the female screw part is formed, and an outer cylindrical body located on an outer side in a radial direction with respect to the inner cylindrical part, the outer cylindrical body provided with an outer cylindrical part on an outer surface of which the locking unit is formed.

According to one embodiment, a rotation control mechanism is provided between the inner cylindrical body and the outer cylindrical body, the rotation control mechanism restricts relative rotation of the outer cylindrical body to one side in a circumferential direction of the adapter body with respect to the inner cylindrical body and permits relative rotation of the outer cylindrical body to the other side in the circumferential direction with respect to the inner cylindrical body.

According to one embodiment, the female screw part of the adapter body is configured to be screwable to the male screw part of the female connector part until a cylindrical part on which the male screw part is formed of the female connector part penetrates the adapter body in an axial direction, or until a distal end of the cylindrical part is flush with an inner edge of one end in the axial direction of the adapter body.

According to one embodiment, the female connector part is provided with the cylindrical part that defines an insertion opening into which a first male luer part of the first male connector part and a second male luer part of the second male connector part are externally insertable, and a valve element that blocks the insertion opening.

According to one embodiment, the locking unit is formed of a recess engageable with a locking claw of the second male connector part.

According to a second embodiment of the present disclosure, a connector set comprises the above-described conversion adapter, and a medical connector provided with the female connector part to which the conversion adapter is attachable.

According to a second embodiment of the present disclosure, an assembly according comprises the above-described conversion adapter, and the female connector part to which the conversion adapter is attached.

According to certain embodiments of the present disclosure, a conversion adapter capable of easily converting a female connector part connectable to a luer lock-type male connector part conforming to ISO 80369 into a female connector part connectable to a male connector part not conforming to ISO 80369 in a locked state using a predetermined lock mechanism, a connector set including the conversion adapter, and an assembly provided with the conversion adapter may be provided.

### BRIEF DESCRIPTION OF DRAWINGS

FIG. 1 is a perspective view illustrating an assembly as an embodiment of the present disclosure;

FIG. 2 is an exploded perspective view of the assembly illustrated in FIG. 1, the diagram illustrating a connector set as an embodiment of the present disclosure;

FIG. 3 is a top view of the assembly illustrated in FIG. 1;

FIG. 4 is a cross-sectional view taken along line I-I in FIG. 3 of the assembly illustrated in FIG. 1;

FIG. 5 is a cross-sectional view taken along line II-II in FIG. 3 of the assembly illustrated in FIG. 1;

FIG. 6 is an exploded perspective view of a conversion adapter as an embodiment of the present disclosure illustrated in FIG. 2;

FIG. 7A is a diagram illustrating a rotation control mechanism of the conversion adapter illustrated in FIG. 2;

FIG. 7B is a diagram illustrating the rotation control mechanism of the conversion adapter illustrated in FIG. 2;

FIG. 8 is a cross-sectional view illustrating a state in which a luer lock-type first male connector part conforming to ISO 80369 in another medical connector is connected to the medical connector illustrated in FIG. 2;

FIG. 9 is a cross-sectional view illustrating a state in which a second male connector part not conforming to ISO 80369 in another medical connector is connected to the assembly illustrated in FIG. 1;

FIG. 10A is a perspective view of a connection state of the assembly and the medical connector illustrated in FIG. 9;

FIG. 10B is a diagram illustrating a variation of the medical connector illustrated in FIG. 2 to which the conversion adapter illustrated in FIG. 2 is attachable; and

FIG. 11 is a diagram illustrating an example of a infusion line including an assembly in which the conversion adapter illustrated in FIG. 2 is attached to the medical connector illustrated in FIG. 10B.

### DETAILED DESCRIPTION

Hereinafter, embodiments of a conversion adapter, a connector set, and an assembly according to the present disclosure are described by way of example with reference to the drawings. In the drawings, the same components are denoted by the same reference numerals.

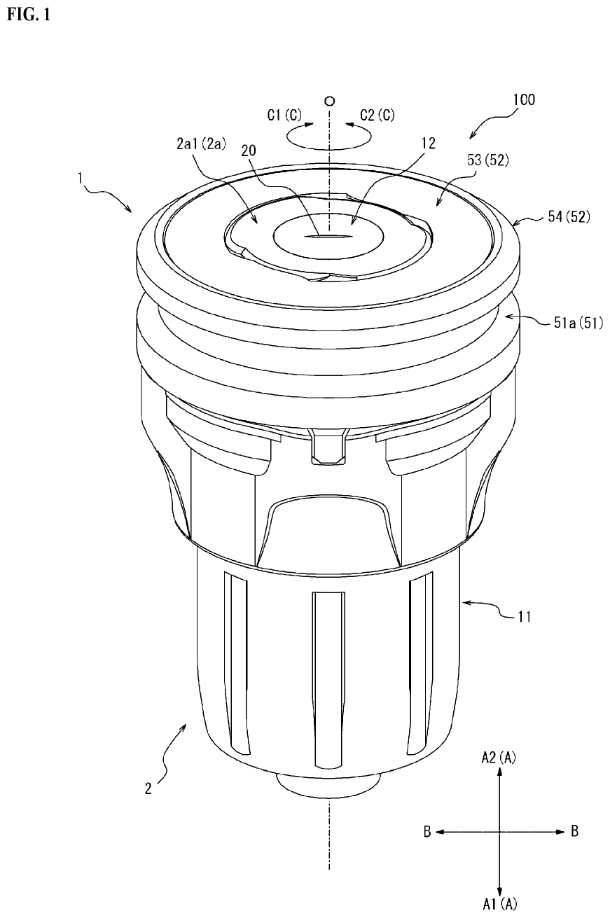

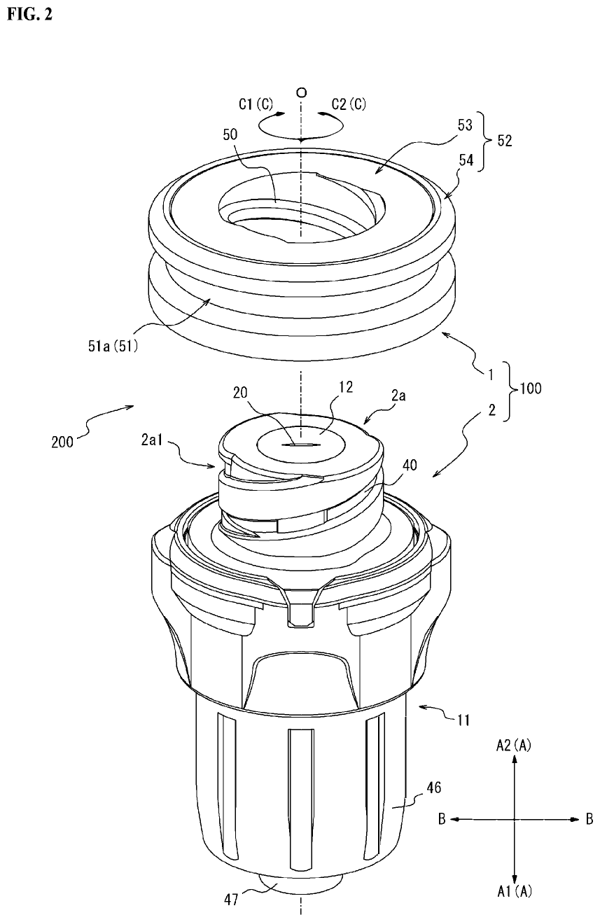

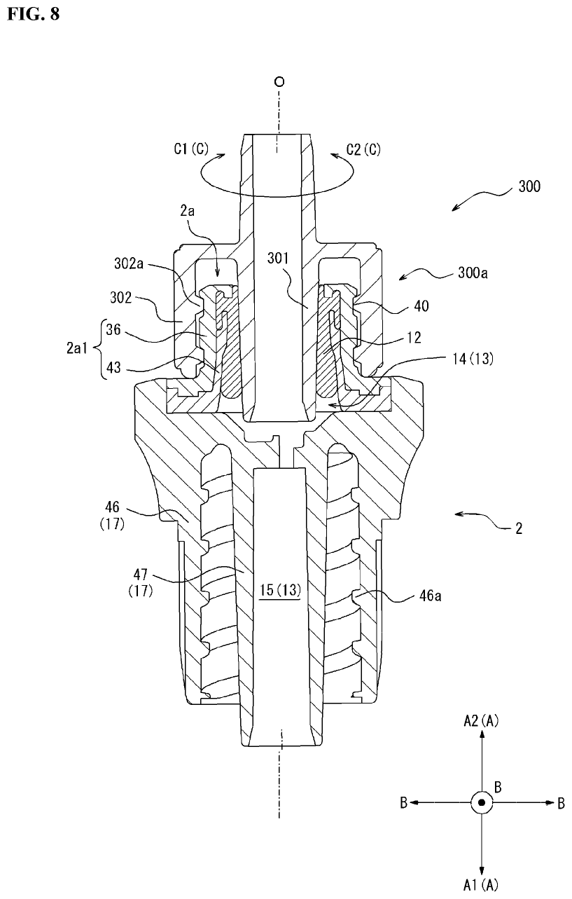

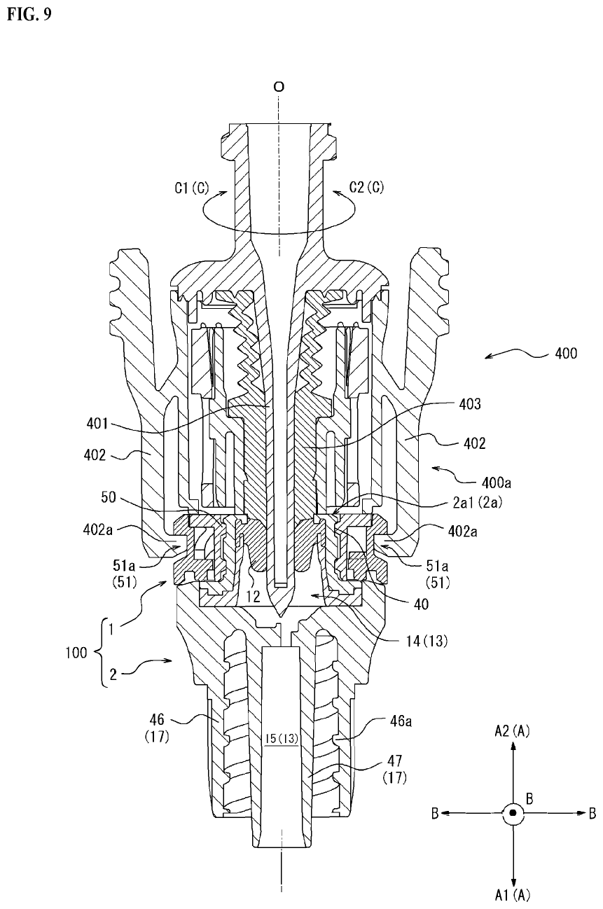

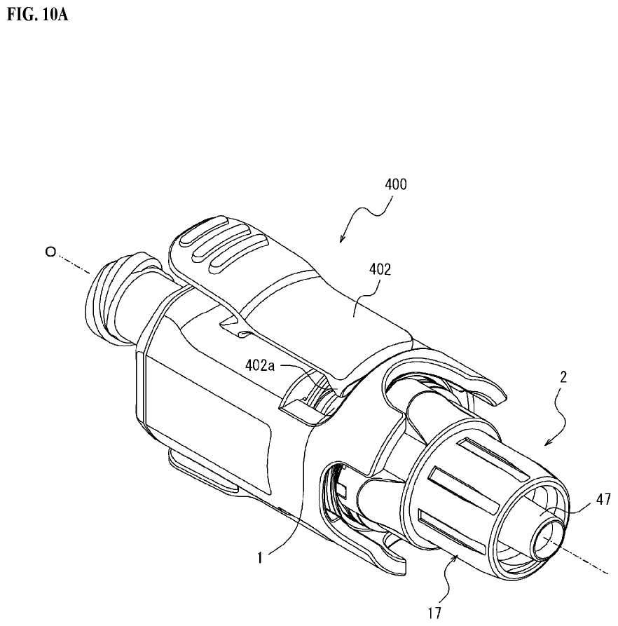

FIG. 1 is a perspective view illustrating an assembly 100 as an embodiment of an assembly according to the present disclosure. FIG. 2 is an exploded perspective view of the assembly 100 illustrated in FIG. 1. In other words, FIG. 2 is a diagram illustrating a connector set 200. As illustrated in FIGS. 1 and 2, the assembly 100 is provided with a conversion adapter 1 as an embodiment of a conversion adapter according to the present disclosure, and a medical connector 2. As illustrated in FIG. 2, the medical connector 2 is provided with a female connector part 2a connectable to a luer lock-type male connector part 300a (refer to FIG. 8) conforming to ISO 80369. Hereinafter, for the purpose of distinguishing from another male connector part 400a (refer to FIG. 9) to be described below, the male connector part 300a (refer to FIG. 8) is referred to as a “first male connector part 300a” for convenience of description. The above-described “luer lock-type male connector part conforming to ISO 80369” is intended to mean a luer lock-type male connector part conforming to ISO 80369-2 to ISO 80369-7. As illustrated in FIG. 1, the conversion adapter 1 is attachable to the female connector part 2a of the medical connector 2. Herein, as illustrated in FIG. 1, the conversion adapter 1 and the medical connector 2 may be distributed in a state of the assembly 100 in which the conversion adapter 1 is attached to the female connector part 2a of the medical connector 2. As illustrated in FIG. 2, the conversion adapter 1 and the medical connector 2 may be distributed together as the connector set 200 in a separate state in which the conversion adapter 1 is not attached to the female connector part 2a of the medical connector 2. Furthermore, the conversion adapter 1 and the medical connector 2 may be distributed separately.

As illustrated in FIGS. 1 and 2, the conversion adapter 1 is attached to the female connector part 2a connectable to the luer lock-type first male connector part 300a (refer to FIG. 8) conforming to ISO 80369. The female connector part 2a is configured to be connectable by screwing to the luer lock-type first male connector part 300a (refer to FIG. 8) conforming to ISO 80369 in a state in which the conversion adapter 1 is not attached thereto. In contrast, the female connector part 2a is configured to be connectable to the male connector part 400a (refer to FIG. 9) not conforming to ISO 80369 by a predetermined lock mechanism in a state in which the conversion adapter 1 is attached thereto. Hereinafter, for the purpose of distinguishing from another first male connector part 300a (refer to FIG. 8) described above, the male connector part 400a (refer to FIG. 9) is referred to as a “second male connector part 400a” for convenience of description. That is, the conversion adapter 1 is an adapter that converts the female connector part 2a connectable to the luer lock-type first male connector part 300a (refer to FIG. 8) conforming to ISO 80369 into a state connectable to the second male connector part 400a (refer to FIG. 9) not conforming to ISO 80369 by a predetermined lock mechanism.

### [Medical Connector 2]

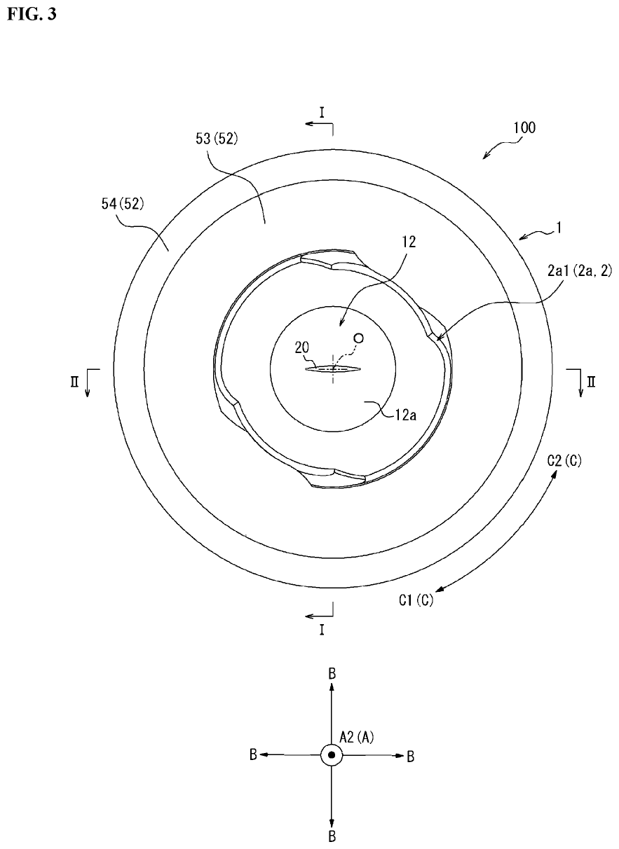

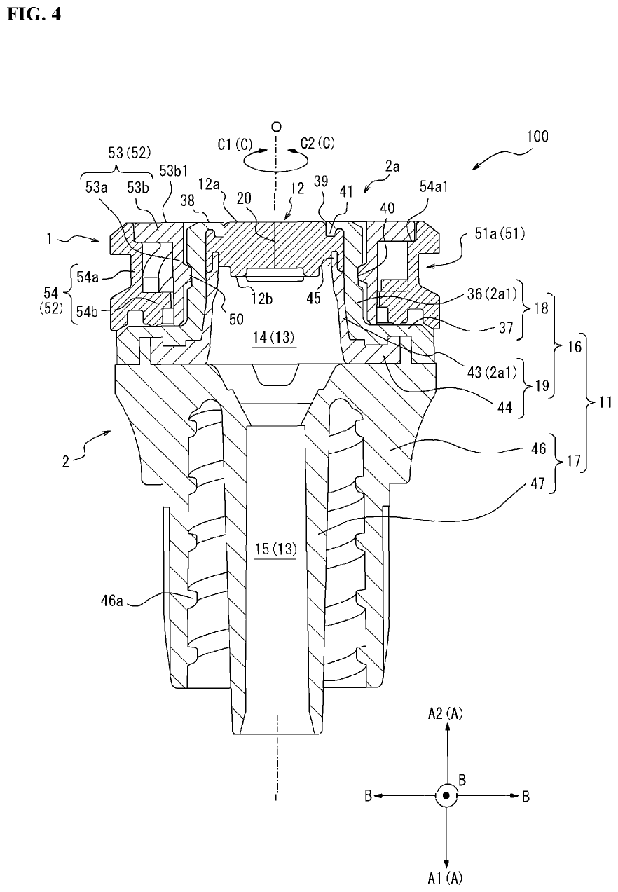

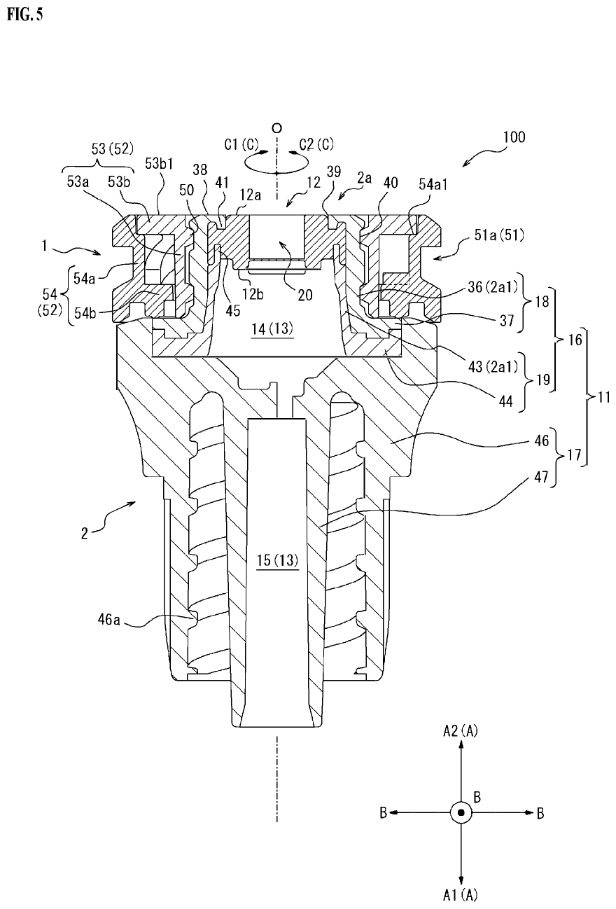

First, a summary of the medical connector 2 including the female connector part 2a to which the conversion adapter 1 is attached is provided with reference to FIGS. 1 to 5. FIG. 3 is a top view of the assembly 100 as seen from a top face 12a side of an elastic valve element 12. FIG. 4 is a cross-sectional view of the assembly 100 taken along line I-I in FIG. 3. FIG. 5 is a cross-sectional view of the assembly 100 taken along line II-II in FIG. 3.

As illustrated in FIGS. 1 to 5, the medical connector 2 is provided with the female connector part 2a connectable to the luer lock-type first male connector part 300a (refer to FIG. 8) conforming to ISO 80369. Specifically, the female connector part 2a is connectable to the first male connector part 300a (refer to FIG. 8) by screwing. The female connector part 2a is at least provided with a cylindrical part 2a1. On an outer surface of the cylindrical part 2a1, a male screw part 40 screwable to a female screw part 302a (refer to FIG. 8) of the luer lock-type first male connector part 300a (refer to FIG. 8) conforming to ISO 80369 is formed. An insertion opening 14 into which a first male luer part 301 (refer to FIG. 8) of the luer lock-type first male connector part 300a (refer to FIG. 8) conforming to ISO 80369 is inserted is defined in the cylindrical part 2a1. In a state in which the conversion adapter 1 is attached to the female connector part 2a, a second male luer part 401 (refer to FIG. 9) of the second male connector part 400a (refer to FIG. 9) not conforming to ISO 80369 is insertable into the insertion opening 14 defined in the cylindrical part 2a1.

More specifically, as illustrated in FIGS. 1 to 5, the medical connector 2 is provided with a housing 11 and the elastic valve element 12 attached to the housing 11. The female connector part 2a of this embodiment is formed of a cap 16 to be described below being a part of the housing 11, and the elastic valve element 12. The cylindrical part 2a1 of the female connector part 2a of this embodiment is described below in detail.

As illustrated in FIGS. 4 and 5, the housing 11 defines a hollow part 13. The elastic valve element 12 is located in the hollow part 13. The hollow part 13 includes the insertion opening 14 into which the first male connector part 300a (refer to FIG. 8) and the second male connector part 400a (refer to FIG. 9) to be described below are externally insertable, and a flow path 15 communicating with the insertion opening 14. The elastic valve element 12 blocks the insertion opening 14 out of the hollow part 13. The phrase “flow path communicating with the insertion opening” is intended to include not only a flow path directly connected to the insertion opening but also a flow path connected to the insertion opening via another space. The flow path 15 of this embodiment is the flow path directly connected to the insertion opening 14.

The housing 11 is provided with the cap 16 that defines the insertion opening 14 into which the first male connector part 300a (refer to FIG. 8) and the second male connector part 400a (refer to FIG. 9) are externally inserted, and a holder 17 that defines the flow path 15 and supports the cap 16.

The cap 16 is provided with a top face cap 18 and a bottom face cap 19. The elastic valve element 12 is compressed and clamped by the top face cap 18 and the bottom face cap 19, and a position thereof in the hollow part 13, more specifically, in the insertion opening 14 is fixed.

The holder 17 is a member that defines the flow path 15 and supports the top face cap 18 and the bottom face cap 19. In this embodiment, both the top face cap 18 and the bottom face cap 19 come into contact with the holder 17 to be directly supported by the holder 17. Note that it is not limited to this configuration. A configuration in which the bottom face cap 19 is held by the top face cap 18, and only the top face cap 18 comes into contact with the holder 17 to be directly supported is also possible. On the other hand, a configuration in which the top face cap 18 is held by the bottom face cap 19, and only the bottom face cap 19 comes into contact with the holder 17 to be directly supported is also possible.

Examples of materials of the holder 17, the top face cap 18, and the bottom face cap 19 of the housing 11 include, for example, various resin materials such as polyolefins such as polyethylene, polypropylene, and an ethylene-propylene copolymer; ethylene-vinyl acetate copolymer (EVA); polyvinyl chloride; polyvinylidene chloride; polystyrene; polyamide; polyimide; polyamideimide; polycarbonate; poly-(4-methylpentene-1); ionomer; acrylic resin; polymethyl methacrylate; acrylonitrile-butadiene-styrene copolymer (ABS resin); acrylonitrile-styrene copolymer (AS resin); butadiene-styrene copolymer; polyesters such as polyethylene terephthalate (PET), polybutylene terephthalate (PBT), and polycyclohexane terephthalate (PCT); polyether; polyetherketone (PEK); polyetheretherketone (PEEK); polyetherimide; polyacetal (POM); polyphenylene oxide; modified polyphenylene oxide; polysulfone; polyether sulfone; polyphenylene sulfide; polyarylate; aromatic polyester (liquid crystal polymer); and polytetrafluoroethylene, polyvinylidene fluoride, and other fluorine-based resins. A blend, a polymer alloy and the like containing one or more of them may be used. In addition, various glass materials, ceramic materials, and metal materials may be used.

The elastic valve element 12 includes a slit 20 so that this may be elastically deformed to be opened and closed when the first male connector part 300a (refer to FIG. 8) and the second male connector part 400a (refer to FIG. 9) are attached to and detached from the medical connector 2. The elastic valve element 12 is arranged so as to block the insertion opening 14 defined by the top face cap 18 and the bottom face cap 19. Specifically, the elastic valve element 12 is clamped by a clamp unit formed of the top face cap 18 and the bottom face cap 19, and a position thereof in the insertion opening 14 is fixed.

The elastic valve element 12 is molded and formed to be elastically deformable. Examples of materials of the elastic valve element 12 include, for example, various rubber materials such as natural rubber, isoprene rubber, butadiene rubber, styrene-butadiene rubber, nitrile rubber, chloroprene rubber, butyl rubber, acrylic rubber, ethylene-propylene rubber, hydrin rubber, urethane rubber, silicone rubber, and fluororubber, and various thermoplastic elastomers such as styrene-based, polyolefin-based, polyvinyl chloride-based, polyurethane-based, polyester-based, polyamide-based, polybutadiene-based, transpolyisoprene-based, fluorine-based, and chlorinated polyethylene-based elastomers, and one or two or more of them may be mixed.

The female connector part 2a of the medical connector 2 of this embodiment is formed of the top face cap 18, the bottom face cap 19, and the elastic valve element 12 clamped by the top face cap 18 and the bottom face cap 19 described above.

As illustrated in FIGS. 4 and 5, the top face cap 18 is provided with a substantially cylindrical hollow cylindrical part 36 and a flange part 37. The flange part 37 protrudes outward in a radial direction B from an end in an insertion direction A1 of the first male connector part 300a (refer to FIG. 8) and the second male connector part 400a (refer to FIG. 9) on one side in an axial direction of the hollow cylindrical part 36.

As illustrated in FIGS. 4 and 5, a planar upper end face 38 extending in a direction orthogonal to the insertion direction A1 and a removal direction A2 is provided on an end in the removal direction A2 of the first male connector part 300a (refer to FIG. 8) and the second male connector part 400a (refer to FIG. 9) on the other side in the axial direction of the hollow cylindrical part 36. The upper end face 38 includes a substantially circular edge 39 that defines one end in the removal direction A2 of the insertion opening 14 into which the first male connector part 300a (refer to FIG. 8) and the second male connector part 400a (refer to FIG. 9) are externally inserted.

On an outer peripheral surface of the hollow cylindrical part 36, a male screw part 40 screwed to the female screw part 302a (refer to FIG. 8) of the luer lock-type first male connector part 300a (refer to FIG. 8) conforming to ISO 80369 is formed. The flange part 37 is a portion integrally molded with the hollow cylindrical part 36, and when the flange part 37 is engaged with the holder 17 to be described below, the top face cap 18 is held by the holder 17.

As illustrated in FIGS. 4 and 5, a locking protrusion 41 protruding in the insertion direction A1 is provided in the vicinity of the edge 39 out of an inner wall of the hollow cylindrical part 36. The locking protrusion 41 enters an annular groove formed on an outer edge of the top face 12a of the elastic valve element 12 described above, and compresses the elastic valve element 12 together with a locking protrusion 45 of the bottom face cap 19.

As illustrated in FIGS. 4 and 5, the bottom face cap 19 is provided with a substantially cylindrical hollow cylindrical part 43 and a flange part 44, similarly to the top face cap 18. The flange part 44 protrudes outward in the radial direction B from an end in the insertion direction A1 of the hollow cylindrical part 43. An end in the removal direction A2 of the hollow cylindrical part 43 enters an annular groove formed on an outer edge of a bottom face 12b of the elastic valve element 12 described above, and forms the locking protrusion 45 that compresses to clamp the elastic valve element 12 together with the locking protrusion 41 of the top face cap 18.

The bottom face cap 19 is held by the top face cap 18 by ultrasonic bonding and the like to an inner surface of the hollow cylindrical part 36 of the top face cap 18 and/or a lower surface (surface on a lower side in FIGS. 4 and 5) of the flange part 37. A lower surface (surface on a lower side in FIGS. 4 and 5) of the flange part 44 of the bottom face cap 19 is supported by the holder 17 to be described below.

As illustrated in FIGS. 4 and 5, the holder 17 supports the top face cap 18 and the bottom face cap 19, and defines the flow path 15 therein. The holder 17 of this embodiment supports both the top face cap 18 and the bottom face cap 19 by directly coming into contact with them, but it is not limited to this configuration as described above. Members of the holder 17 in direct contact with the top face cap 18 and the bottom face cap 19 are preferably bonded by, for example, ultrasonic bonding and the like.

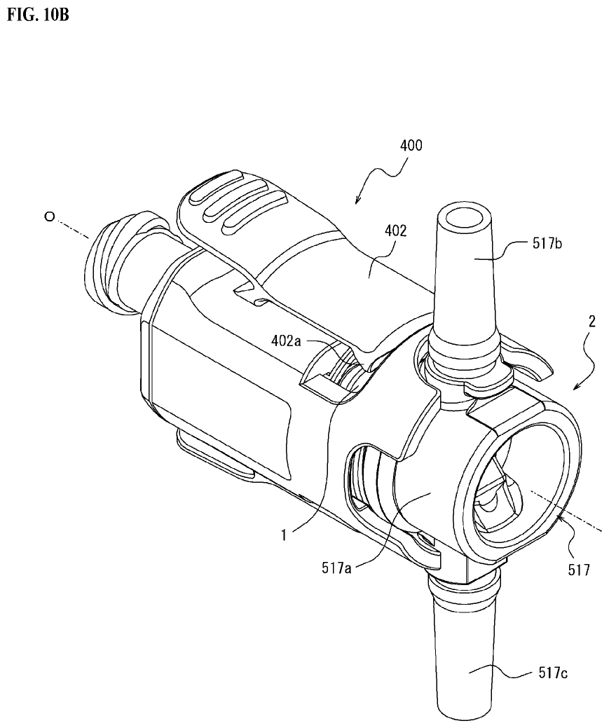

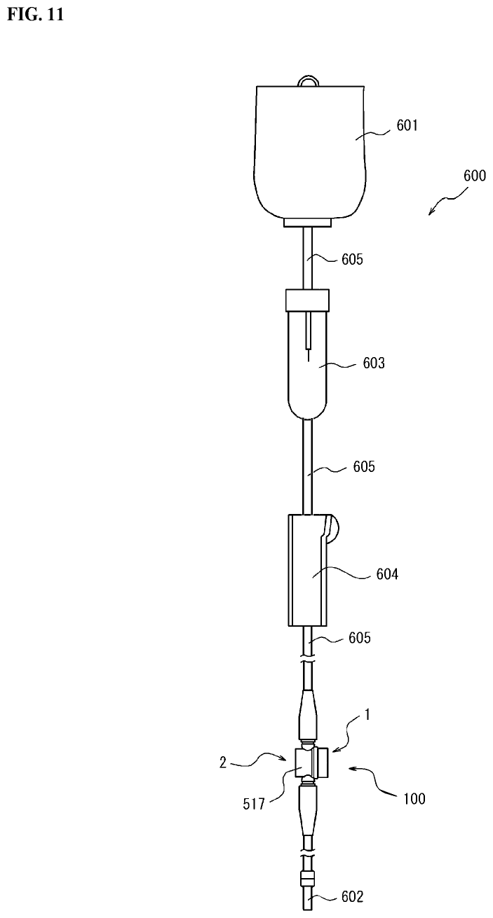

As illustrated in FIGS. 4 and 5, the holder 17 of this embodiment is provided with the luer lock-type first male connector part conforming to ISO 80369. Specifically, the holder 17 of this embodiment is provided with a substantially cylindrical cylindrical part 46 on which a female screw part 46a screwable to the male screw part 40 of the female connector part 2a of another medical connector and the like is formed, and a male luer part 47 provided in a hollow part defined by the cylindrical part 46. Note that a shape of the holder 17 of the medical connector 2 is not limited to the configuration of this embodiment. The medical connector 2 may be provided with a holder 517 as illustrated in FIG. 10B, for example. The holder 517 is provided with a substantially cylindrical holder body 517a defining a hollow part therein, and cylindrical upstream port part 517b and downstream port part 517c protruding from an outer peripheral surface of the holder body 517a. The hollow part inside the holder body 517a serves as a part of a flow path from the upstream port part 517b to the downstream port part 517c.

As described above, the female connector part 2a of the medical connector 2 is formed of the cap 16 and the elastic valve element 12. In this embodiment, the cylindrical part 2a1 of the female connector part 2a is formed of the hollow cylindrical part 36 of the top face cap 18 and the hollow cylindrical part 43 of the bottom face cap 19. The male screw part 40 formed on an outer surface of the hollow cylindrical part 36 of the top face cap 18 is screwable to the female screw part 302a (refer to FIG. 8) of the luer lock-type first male connector part 300a (refer to FIG. 8) conforming to ISO 80369. The first male luer part 301 (refer to FIG. 8) of the luer lock-type first male connector part 300a (refer to FIG. 8) conforming to ISO 80369 is externally insertable into the insertion opening 14 inside the hollow cylindrical part 36 of the top face cap 18 and the hollow cylindrical part 43 of the bottom face cap 19.

As described above, the medical connector 2 of this embodiment is provided with the housing 11 and the elastic valve element 12, and the female connector part 2a is formed of a part of the housing 11 and the elastic valve element 12, but it is not limited to this configuration. The medical connector 2 may be provided with the luer lock-type male connector part conforming to ISO 80369 such as the first male connector part 300a (refer to FIG. 8) described in this embodiment and the female connector part 2a connectable thereto, and the specific number of members and the specific shape of members are not especially limited. The female connector part 2a may be, for example, a luer lock-type female connector part conforming to ISO 80369.

### [Conversion Adapter 1]

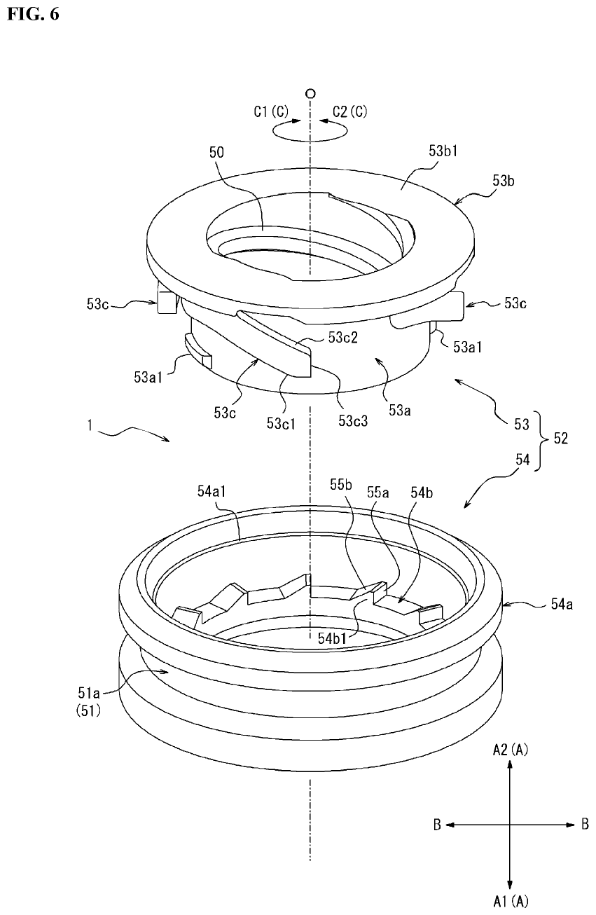

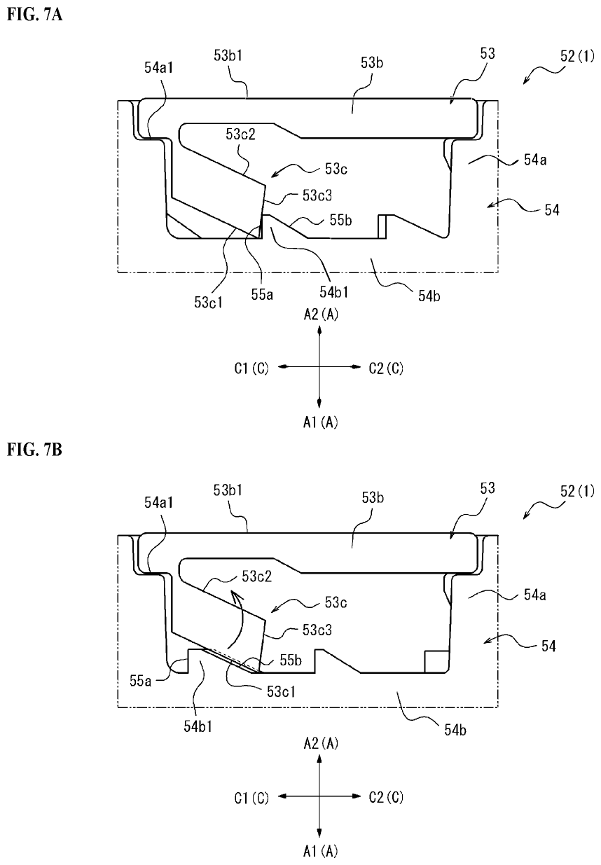

Next, the conversion adapter 1 attachable to the female connector part 2a is described with reference to FIGS. 1 to 7. FIG. 6 is an exploded perspective view of the conversion adapter 1 illustrated in FIG. 2. FIGS. 7A and 7B are diagrams illustrating a rotation control mechanism of the conversion adapter 1.

As illustrated in FIGS. 1 to 5, the conversion adapter 1 is attachable to the female connector part 2a connectable to the luer lock-type first male connector part 300a (refer to FIG. 8) conforming to ISO 80369. Specifically, as illustrated in FIG. 2 and the like, the conversion adapter 1 is provided with a female screw part 50 screwable to the male screw part 40 of the female connector part 2a. The conversion adapter 1 is connected to the female connector part 2a by screw joining with the female screw part 50 screwed to the male screw part 40.

Because the conversion adapter 1 is provided with the female screw part 50 screwable to the male screw part 40 of the female connector part 2a connectable to the luer lock-type first male connector part 300a (refer to FIG. 8) conforming to ISO 80369, the conversion adapter 1 may be easily connected to the female connector part 2a.

The conversion adapter 1 is provided with a locking unit 51 capable of locking the second male connector part 400a (refer to FIG. 9) not conforming to ISO 80369. As is described below in detail, the locking unit 51 of this embodiment is formed of an annular groove 51a as a recess engageable with a locking claw 402 (refer to FIG. 9) of the second male connector part 400a (refer to FIG. 9).

Because the conversion adapter 1 itself is provided with the locking unit 51, the second male connector part 400a (refer to FIG. 9) not conforming to ISO 80369 may be locked using a predetermined lock mechanism using the locking unit 51 of the conversion adapter 1 regardless of a difference in shape of a part other than the female connector part 2a in a medical device such as the medical connector 2 provided with the female connector part 2a.

Hereinafter, the conversion adapter 1 of this embodiment is described in further detail.

As illustrated in FIGS. 1 and 2, the conversion adapter 1 of this embodiment is provided with a cylindrical adapter body 52. As illustrated in FIGS. 2 and 4 to 6, the female screw part 50 of the conversion adapter 1 of this embodiment is formed on an inner surface of the adapter body 52. As illustrated in FIGS. 1, 2 and 4 to 6, the locking unit 51 of the conversion adapter 1 of this embodiment is formed on an outer surface of the adapter body 52. With such a configuration, the conversion adapter 1 may have a simple configuration.

The conversion adapter 1 of this embodiment is formed only of the cylindrical adapter body 52, but it is not limited to this configuration. The conversion adapter 1 may be provided with another portion in addition to the cylindrical adapter body 52.

Hereinafter, for convenience of description, an axial direction of the cylindrical adapter body 52 is referred to as an “axial direction A of the adapter body 52” or simply as an “axial direction A”. The axial direction A of the adapter body 52 is substantially parallel to the insertion direction A1 and the removal direction A2 of the female connector part 2a in a state in which the adapter body 52 is connected to the female connector part 2a. Therefore, hereinafter, one side in the axial direction A is referred to as the insertion direction A1, and the other side in the axial direction A is referred to as the removal direction A2. A radial direction of a circle around a central axis O of the adapter body 52 is referred to as a “radial direction B of the adapter body 52” or simply as a “radial direction B”. Furthermore, a circumferential direction around the central axis O of the adapter body 52 is referred to as a “circumferential direction C of the adapter body 52” or simply as a “circumferential direction C”. The central axis O, the radial direction B, and the circumferential direction C of the adapter body 52 coincide with a central axis, a radial direction, and a circumferential direction of the cylindrical part 2a1 of the female connector part 2a in a state in which the conversion adapter 1 is attached to the female connector part 2a. Therefore, in the drawings, for convenience of description, the central axis of the cylindrical part 2a1 of the female connector part 2a is also denoted by reference character “O”, the radial direction of the cylindrical part 2a1 is also denoted by reference character “B”, and the circumferential direction of the cylindrical part 2a1 is also denoted by reference character “C”.

As illustrated in FIG. 6, the adapter body 52 of this embodiment is provided with an inner cylindrical body 53 and an outer cylindrical body 54. The inner cylindrical body 53 is provided with an inner cylindrical part 53a on an inner surface of which the female screw part 50 is formed. The outer cylindrical body 54 is provided with an outer cylindrical part 54a located on an outer side in the radial direction B with respect to the inner cylindrical part 53a, the outer cylindrical part 54a on an outer surface of which the locking unit 51 is formed.

As illustrated in FIG. 6, the inner cylindrical body 53 of this embodiment is provided with the inner cylindrical part 53a on the inner surface of which the female screw part 50 is formed as described above, an annular flange part 53b protruding outward in the radial direction B from an end in the removal direction A2 of the inner cylindrical part 53a, and a plurality of protrusions 53c protruding from the flange part 53b in the insertion direction A1.

A support protrusion 53a1 that supports the outer cylindrical body 54 from an insertion direction A1 side is provided in a protruding manner on an end in the insertion direction A1 of the inner cylindrical part 53a. Although a plurality of (two in this embodiment) support protrusions 53a1 is provided at positions separated from each other in the circumferential direction C, the number and positions thereof are not especially limited. The support protrusion 53a1 of this embodiment sandwiches the outer cylindrical body 54 in the axial direction A together with the flange part 53b, thereby restricting relative movement of the outer cylindrical body 54 in the axial direction A with respect to the inner cylindrical body 53. Specifically, the relative movement of the outer cylindrical body 54 in the insertion direction A1 with respect to the inner cylindrical body 53 is restricted by the flange part 54b to be described below of the outer cylindrical body 54 abutting the support protrusion 53a1 of the inner cylindrical part 53a of the inner cylindrical body 53. The relative movement of the outer cylindrical body 54 in the removal direction A2 with respect to the inner cylindrical body 53 is restricted by the outer cylindrical part 54a of the outer cylindrical body 54 abutting the flange part 53b of the inner cylindrical body 53. In this manner, the inner cylindrical body 53 and the outer cylindrical body 54 of this embodiment may be coupled to each other so as not to be separated from each other in the axial direction A. Therefore, the inner cylindrical body 53 and the outer cylindrical body 54 are inhibited from being unintentionally separated from each other, and operability for a medical worker who uses the conversion adapter 1 may be improved.

The plurality of protrusions 53c is arranged at predetermined intervals in the circumferential direction C. As the plurality of protrusions 53c of this embodiment, four protrusions 53c are arranged at regular intervals in the circumferential direction C. Each protrusion 53c is located on an outer side in the radial direction B with respect to the inner cylindrical part 53a. The protrusions 53c are arranged outward in the radial direction B from the outer surface of the inner cylindrical part 53a so as to be separated from each other.

At described below in detail, the protrusion 53c forms a part of the rotation control mechanism of this embodiment (refer to FIGS. 7A and 7B). The inner cylindrical body 53 of this embodiment is provided with a plurality of protrusions 53c, but the number thereof is not especially limited. The number of the protrusions 53c of the inner cylindrical body 53 may be only one or plural other than four.

More specifically, each protrusion 53c of this embodiment is formed of a rod-shaped protrusion protruding from the flange part 53b in a direction inclined with respect to the insertion direction A1. That is, the rod-shaped protrusion as the protrusion 53c of this embodiment protrudes from the flange part 53b in the direction inclined with respect to the axial direction A. Therefore, as illustrated in FIGS. 7A and 7B, a lower side surface 53c1 being a side surface on the insertion direction A1 side of the protrusion 53c and an upper side surface 53c2 being a side surface on the removal direction A2 side of the protrusion 53c are formed of inclined surfaces inclined with respect to the axial direction A. As illustrated in FIGS. 7A and 7B, a distal end face 53c3 of the protrusion 53c is formed of a plane substantially parallel to the axial direction A.

Each protrusion 53c is swingable by being elastically deformed in the axial direction A with a proximal end connected to the flange part 53b as a fulcrum. As described below in detail, the rotation control mechanism of this embodiment restricts or permits the relative rotation of the inner cylindrical body 53 and the outer cylindrical body 54 with the protrusion 53c of the inner cylindrical body 53 swinging in the axial direction A according to an engagement relationship with a convex part 54b1 of the outer cylindrical body 54 (refer to FIGS. 7A and 7B).

The outer cylindrical body 54 is provided with the outer cylindrical part 54a on the outer surface of which the locking unit 51 is formed as described above, and an annular flange part 54b protruding inward in the radial direction B from an end in the insertion direction A1 of the outer cylindrical part 54a.

As described above, the locking unit 51 formed on the outer surface of the outer cylindrical part 54a is the annular groove 51a as the recess extending across an entire area in the circumferential direction C. Note that the locking unit 51 is not limited to the annular groove 51a of this embodiment. The locking unit 51 may be formed of, for example, a plurality of recesses arranged so as to be separated from each other in the circumferential direction C. The locking unit 51 may be appropriately designed according to a specific shape of the second male connector part 400a (refer to FIG. 9), and is not limited to the recess, but is preferably a recess engageable with the locking claw 402 (refer to FIG. 9) of the second male connector part 400a (refer to FIG. 9) as with the annular groove 51a of this embodiment. In this manner, the locking unit 51 may be easily implemented with a simple configuration.

A stepped surface 54a1 that supports the flange part 53b of the inner cylindrical body 53 from the insertion direction A1 side is preferably formed on an inner surface of the outer cylindrical part 54a. The outer cylindrical part 54a of this embodiment has different inner diameters between the insertion direction A1 side and the removal direction A2 side. The stepped surface 54a1 of this embodiment is an annular plane facing in the removal direction A2 that connects parts having different inner diameters described above of the inner surface of the outer cylindrical part 54a. By providing such stepped surface 54a1, the inner cylindrical body 53 and the outer cylindrical body 54 may be coupled to each other in a state in which the flange part 53b forming the end in the removal direction A2 of the inner cylindrical body 53 is accommodated in the outer cylindrical part 54a. That is, the inner cylindrical body 53 and the outer cylindrical body 54 may be coupled to each other in a state in which the outer cylindrical body 54 covers the outer side in the radial direction B of the inner cylindrical body 53 so that the inner cylindrical body 53 cannot be clamped from the outer side in the radial direction B. Therefore, it is possible to inhibit the inner cylindrical body 53 from rotating so as to be detached from the female connector part 2a in a state in which the inner cylindrical body 53 is connected to the female connector part 2a by screw joining. This is described below in detail.

A plurality of convex parts 54b1 is provided on a surface on the removal direction A2 side of the flange part 54b. The plurality of convex parts 54b1 is arranged at predetermined intervals in the circumferential direction C. As the plurality of convex parts 54b1 of this embodiment, 12 protrusions 54b1 are arranged at regular intervals in the circumferential direction C. As described below in detail, the convex part 54b1 forms a part of the rotation control mechanism of this embodiment (refer to FIGS. 7A and 7B). The outer cylindrical body 54 of this embodiment is provided with a plurality of convex parts 54b1, but the number thereof is not especially limited. The number of the convex parts 54b1 of the outer cylindrical body 54 may be only one or plural other than 12.

More specifically, as illustrated in FIGS. 6, 7A, and 7B, each convex part 54b1 of this embodiment is provided with a rotation restricting surface 55a formed of a plane substantially parallel to the axial direction A on one side in the circumferential direction C. Each convex part 54b1 of this embodiment is provided with a rotation permitting surface 55b formed of a plane inclined with respect to the axial direction A on the other side in the circumferential direction C. The convex part 54b1 of this embodiment is a substantially triangular protrusion having the rotation restricting surface 55a and the rotation permitting surface 55b as both side surfaces, but the shape thereof is not especially limited as long as rotation control described below is possible.

Next, the rotation control mechanism of the conversion adapter 1 of this embodiment is described. The rotation control mechanism is provided between the inner cylindrical body 53 and the outer cylindrical body 54 of this embodiment. The rotation control mechanism restricts the outer cylindrical body 54 from relatively rotating to one side in the circumferential direction C with respect to the inner cylindrical body 53. In contrast, the rotation control mechanism permits the outer cylindrical body 54 from relatively rotating to the other side in the circumferential direction C with respect to the inner cylindrical body 53. By providing such rotation control mechanism, it is possible to inhibit the screw joining between the female connector part 2a of the medical connector 2 and the inner cylindrical body 53 from being loosened by the rotation of the outer cylindrical body 54.

Specifically, in the conversion adapter 1 of this embodiment, the female screw part 50 of the inner cylindrical body 53 rotates in a tightening direction C1 that is one side in the circumferential direction C and is screwed to the male screw part 40 of the female connector part 2a to be joined by screw joining to the medical connector 2. Conversely, in a state in which the conversion adapter 1 and the medical connector 2 are joined by screw joining, when the female screw part 50 of the inner cylindrical body 53 rotates in a loosening direction C2 that is the other side in the circumferential direction C with respect to the male screw part 40 of the female connector part 2a, the screw joining between the conversion adapter 1 and the medical connector 2 is loosened.

The rotation control mechanism restricts the outer cylindrical body 54 from relatively rotating in the tightening direction C1 with respect to the inner cylindrical body 53. That is, when the outer cylindrical body 54 is about to relatively rotate in the tightening direction C1 with respect to the inner cylindrical body 53, the rotation of the outer cylindrical body 54 is restricted by the inner cylindrical body 53 and this does not relatively rotate. Therefore, when the conversion adapter 1 is connected to the female connector part 2a of the medical connector 2, the outer cylindrical body 54 is rotated in the tightening direction C1. As a result, the inner cylindrical body 53 and the outer cylindrical body 54 rotate together by the rotation control mechanism, and the female screw part 50 of the inner cylindrical body 53 is joined by screw joining to the male screw part 40 of the female connector part 2a.

In a state in which the conversion adapter 1 and the medical connector 2 are joined by screw joining, the rotation control mechanism permits the outer cylindrical body 54 to relatively rotate in the loosening direction C2 with respect to the inner cylindrical body 53. That is, when the outer cylindrical body 54 is about to relatively rotate in the loosening direction C2 with respect to the inner cylindrical body 53, the rotation of the outer cylindrical body 54 is not restricted by the inner cylindrical body 53 and this may relatively rotate. Therefore, when the outer cylindrical body 54 is rotated in the loosening direction C2 in a state in which the conversion adapter 1 is connected to the female connector part 2a of the medical connector 2, the outer cylindrical body 54 idles with respect to the inner cylindrical body 53 by the rotation control mechanism. Therefore, even if an unintended external force acts on the outer cylindrical body 54 exposed to the outside in a state in which the conversion adapter 1 is connected to the female connector part 2a of the medical connector 2, the screw joining between the conversion adapter 1 and the female connector part 2a of the medical connector 2 is unlikely to be loosened. As a result, it is possible to inhibit the conversion adapter 1 from being unintentionally detached from the female connector part 2a of the medical connector 2.

Hereinafter, the rotation control mechanism of this embodiment is described in further detail. As illustrated in FIGS. 7A and 7B, the rotation control mechanism of this embodiment is a ratchet mechanism. The rotation control mechanism of this embodiment is formed of the protrusion 53c of the inner cylindrical body 53 and the convex part 54b1 of the outer cylindrical body 54.

In a state in which the inner cylindrical body 53 is coupled to the outer cylindrical body 54, the distal end face 53c3 of the protrusion 53c of the inner cylindrical body 53 and the rotation restricting surface 55a of the convex part 54b1 of the outer cylindrical body 54 are arranged so as to be opposed to each other in the circumferential direction C.

FIG. 7A illustrates a state in which the outer cylindrical body 54 is about to relatively rotate with respect to the inner cylindrical body 53 in the tightening direction C1 (to the left in FIGS. 7A and 7B). As illustrated in FIG. 7A, when the outer cylindrical body 54 is about to relatively rotate with respect to the inner cylindrical body 53 in the tightening direction C1, the rotation restricting surface 55a of the convex part 54b1 abuts the distal end face 53c3 of the protrusion 53c of the inner cylindrical body 53 to press the inner cylindrical body 53 in the tightening direction C1. That is, it is possible to rotate the inner cylindrical body 53 and the outer cylindrical body 54 together by trying to relatively rotate the outer cylindrical body 54 with respect to the inner cylindrical body 53 in the tightening direction C1 (to the left in FIG. 7A).

In contrast, FIG. 7B illustrates a state in which the outer cylindrical body 54 is about to relatively rotate with respect to the inner cylindrical body 53 in the loosening direction C2 (to the right in FIGS. 7A and 7B). As illustrated in FIG. 7B, when the outer cylindrical body 54 is about to relatively rotate with respect to the inner cylindrical body 53 in the loosening direction C2, the rotation restricting surface 55a of the convex part 54b1 does not abut the distal end face 53c3 of the protrusion 53c of the inner cylindrical body 53. When the lower side surface 53c1 of the protrusion 53c slides on the rotation permitting surface 55b of the convex part 54b1, the protrusion 53c is elastically deformed toward the removal direction A2 side with the proximal end connected to the flange part 53b as a fulcrum to swing (refer to arrow in FIG. 7B). Therefore, the protrusion 53c may get over the convex part 54b1. That is, even when the outer cylindrical body 54 is relatively rotated with respect to the inner cylindrical body 53 in the loosening direction C2 (to the right in FIGS. 7A and 7B), the inner cylindrical body 53 does not rotate together with the outer cylindrical body 54, and the outer cylindrical body 54 idles with respect to the inner cylindrical body 53.

Note that the rotation control mechanism is not limited to the configuration of this embodiment. In this embodiment, the inner cylindrical body 53 is provided with the protrusion 53c, and the outer cylindrical body 54 is provided with the convex part 54b1; however, the inner cylindrical body 53 may be provided with a convex part having a shape similar to that of the convex part 54b1, and the outer cylindrical body 54 may be provided with a protrusion having a shape similar to that of the protrusion 53c. The shapes of the protrusion 53c and the convex part 54b1 are not limited to the shapes of this embodiment. Furthermore, the ratchet mechanism is not required as long as the above-described rotation control may be executed. Note that, by using the ratchet mechanism as in this embodiment, the above-described rotation control may be easily implemented with a simple configuration.

Herein, as illustrated in FIGS. 1, 4, and 5, the female screw part 50 of the adapter body 52 of this embodiment is configured to be screwable to the male screw part 40 of the female connector part 2a until a distal end of the cylindrical part 2a1 of the female connector part 2a is flush with an inner edge of an end on the removal direction A2 side, which is an inner edge of one end in the axial direction A, of the adapter body 52. That is, the upper end face 38 forming the distal end of the cylindrical part 2a1 of the female connector part 2a of this embodiment is flush with an inner edge of the upper surface 53b1 of the flange part 53b of the inner cylindrical body 53 forming the inner edge of the end on the removal direction A2 side of the adapter body 52 in a state in which the conversion adapter 1 is connected to the female connector part 2a (refer to FIGS. 1, 4, and 5). In this manner, because the distal end of the cylindrical part 2a1 of the female connector part 2a is exposed from the conversion adapter 1, the distal end of the cylindrical part 2a1 may be easily wiped, and a wiping property of the cylindrical part 2a1 may be improved.

The female screw part 50 of the adapter body 52 may be configured to be screwable to the male screw part 40 of the female connector part 2a until the cylindrical part 2a1 of the female connector part 2a penetrates the adapter body 52 in the axial direction A. In this manner, the wiping property of the cylindrical part 2a1 may be improved as in the description above.

[Connection Between Female Connector Part 2a and First Male Connector Part 300a]

FIG. 8 is a cross-sectional view illustrating a state in which the luer lock-type first male connector part 300a conforming to ISO 80369 in another medical connector 300 is connected to the female connector part 2a in the medical connector 2.

As illustrated in FIG. 8, the first male connector part 300a of the medical connector 300 is provided with the first male luer part 301 and a cylindrical part 302 surrounding an outer side in a radial direction of the first male luer part 301. The female screw part 302a is formed on an inner surface of the cylindrical part 302.

As illustrated in FIG. 8, the conversion adapter 1 is not connected to the female connector part 2a of the medical connector 2. As illustrated in FIG. 8, the first male luer part 301 of the first male connector part 300a is externally inserted into the insertion opening 14 of the cylindrical part 2a1 of the female connector part 2a. In this state, the male screw part 40 of the cylindrical part 2a1 of the female connector part 2a is screwed to the female screw part 302a of the first male connector part 300a, so that the female connector part 2a and the first male connector part 300a are connected to each other.

[Connection Between Female Connector Part 2a and Second Male Connector Part 400a]

FIG. 9 is a cross-sectional view illustrating a state in which the second male connector part 400a not conforming to ISO 80369 in another medical connector 400 is connected to the female connector part 2a in the medical connector 2 in a state in which the conversion adapter 1 is connected thereto.

As illustrated in FIG. 9, the second male connector part 400a of the medical connector 400 is provided with the second male luer part 401 and the locking claw 402 located on an outer side in a radial direction of the second male luer part 401.

As illustrated in FIG. 9, the second male luer part 401 of the second male connector part 400a is externally inserted into the insertion opening 14 of the cylindrical part 2a1 of the female connector part 2a. In this state, a distal end protrusion 402a of the locking claw 402 of the second male connector part 400a fits into the annular groove 51a, which is the locking unit 51 of the conversion adapter 1 connected to the female connector part 2a, so that the female connector part 2a to which the conversion adapter 1 is connected and the second male connector part 400a are connected to each other.

Especially, in this embodiment, an attaching operation of the male connector part to the female connector part 2a becomes easy by using the conversion adapter 1. Specifically, as illustrated in FIG. 8, when the first male connector part 300a is connected to the female connector part 2a, the first male connector part 300a is relatively rotated in the circumferential direction of the cylindrical part 2a1 of the female connector part 2a to be joined thereto by screw joining. In contrast, as illustrated in FIG. 9, when the second male connector part 400a is connected to the female connector part 2a, the second male connector part 400a is brought close to the female connector part 2a in the insertion direction A1, and the second male luer part 401 is inserted into the insertion opening 14. Then, by directly pushing the second male connector part 400a in the insertion direction A1, the locking claw 402 of the second male connector part 400a is engaged with the annular groove 51a serving as the locking unit 51 of the conversion adapter 1, and the connection between the female connector part 2a and the second male connector part 400a is completed. That is, the female connector part 2a and the second male connector part 400a may be connected to each other in a predetermined locked state by the locking claw 402 and the annular groove 51a only by the operation of relatively moving the second male connector part 400a with respect to the female connector part 2a in the insertion direction A1.

The second male connector part 400a illustrated in FIG. 9 is a so-called “closed male connector part” including a cylindrical valve element 403 capable of covering the second male luer part 401, but the second male connector part connectable in a predetermined locked state by using the conversion adapter 1 is not limited to the configuration illustrated in FIG. 9. That is, the second male connector part of another shape not conforming to ISO 80369 is also possible.

FIGS. 10A and 10B are diagrams illustrating two types of medical connectors 2 provided with the female connector part 2a with different holder shapes. FIGS. 10A and 10B illustrate a state in which the medical connector 400 illustrated in FIG. 9 is connected to the two types of medical connectors 2. The medical connector 2 illustrated in FIG. 10A is similar to the configuration illustrated in FIGS. 1 to 5, 8, and 9. In contrast, the medical connector 2 illustrated in FIG. 10B is provided with the holder 517 including the substantially cylindrical holder body 517a, the upstream port part 517b, and the downstream port part 517c.

Because the conversion adapter 1 itself is provided with the locking unit 51 as described above, the second male connector part 400a not conforming to ISO 80369 may be locked using a predetermined lock mechanism using the locking unit 51 of the conversion adapter 1 regardless of a difference in shape of a part other than the female connector part 2a in a medical device such as the medical connector 2 provided with the female connector part 2a (for example, difference in holder shape in FIGS. 10A and 10B).

Finally, an example of an infusion line 600 including the conversion adapter 1 and the medical connector 2 is described with reference to FIG. 11.

The infusion line 600 is provided with a drip chamber 603 and a clamp 604 between an infusion bag 601 containing liquid such as a medicinal solution and an indwelling needle 602 inserted into the vein and the like of a patient, and they are connected to each other by a medical tube 605. The medical connector 2 is provided on the infusion line 600 to make the luer lock-type first male connector part 300a (refer to FIG. 8) conforming to ISO 80369 to be connectable to the infusion line 600.

FIG. 11 illustrates a state in which the conversion adapter 1 is attached to the female connector part 2a (refer to FIG. 2 and the like) of the medical connector 2 (assembly state). In this embodiment, by attaching the conversion adapter 1 to the female connector part 2a, the luer lock-type second male connector part 400a (refer to FIG. 9) not conforming to ISO 80369 is connectable to the infusion line 600. Although FIG. 11 illustrates the infusion line 600 including the medical connector 2 illustrated in FIG. 10B, the infusion line 600 may include the medical connector 2 illustrated in FIG. 2 and the like.

The conversion adapter, the connector set, and the assembly according to the present disclosure are not limited to the specific configurations described with respect to the above embodiments, and various variations, changes, and combinations are possible without departing from the scope of claims. For example, the connector set 200 illustrated in FIG. 2 may include another medical device such as the medical tube 605 illustrated in FIG. 11 in addition to the conversion adapter 1 and the medical connector 2. That is, the connector set 200 may be the infusion set including the conversion adapter 1 and the medical connector 2.

Furthermore, in the above-described embodiment, a configuration in which the conversion adapter 1 is attached to the medical connector 2 is described as the assembly 100, but the assembly 100 is not limited to this configuration. The assembly 100 may be configured to be provided with the conversion adapter 1 and the female connector part 2a to which the conversion adapter 1 is attached, and the medical device provided with the female connector part 2a is not limited to the medical connector 2.

The present disclosure relates to a conversion adapter, a connector set, and an assembly.

What is claimed is:

1. A conversion adapter attachable to a female connector part comprising a male screw part to which a luer lock-type first male connector part conforming to ISO 80369 is connectable, the conversion adapter comprising:
a female screw part screwable to the male screw part of the female connector part; and
a locking unit configured to lock to a second male connector part not conforming to ISO 80369.

2. The conversion adapter according to claim 1, comprising:
a cylindrical adapter body; wherein:
the female screw part is formed on an inner surface of the adapter body; and
the locking unit is formed on an outer surface of the adapter body.

3. The conversion adapter according to claim 2, wherein:
the adapter body comprises:
an inner cylindrical body comprising an inner cylindrical part on an inner surface of which the female screw part is formed, and
an outer cylindrical body located on an outer side in a radial direction with respect to the inner cylindrical part, the outer cylindrical body comprising an outer cylindrical part on an outer surface of which the locking unit is formed.

4. The conversion adapter according to claim 3, wherein:
a rotation control mechanism is provided between the inner cylindrical body and the outer cylindrical body, the rotation control mechanism restricting relative rotation of the outer cylindrical body to one side in a circumferential direction of the adapter body with respect to the inner cylindrical body and permitting relative rotation of the outer cylindrical body to the other side in the circumferential direction with respect to the inner cylindrical body.

5. The conversion adapter according to claim 2, wherein:
the female screw part of the adapter body is configured to be screwable to the male screw part of the female connector part until either (i) a cylindrical part on which the male screw part is formed of the female connector part penetrates the adapter body in an axial direction, or (ii) a distal end of the cylindrical part is flush with an inner edge of one end in the axial direction of the adapter body.

6. The conversion adapter according to claim 2, wherein:
the female screw part of the adapter body is configured to be screwable to the male screw part of the female connector part until a cylindrical part on which the male screw part is formed of the female connector part penetrates the adapter body in an axial direction.

7. The conversion adapter according to claim 2, wherein:
the female screw part of the adapter body is configured to be screwable to the male screw part of the female connector part until a distal end of the cylindrical part is flush with an inner edge of one end in the axial direction of the adapter body.

8. The conversion adapter according to claim 5, wherein:
the female connector part comprises:
the cylindrical part, which defines an insertion opening into which a first male luer part of the first male connector part and a second male luer part of the second male connector part are externally insertable, and
a valve element that blocks the insertion opening.

9. The conversion adapter according to claim 1, wherein:
the locking unit comprises a recess engageable with a locking claw of the second male connector part.

10. A connector set comprising:
the conversion adapter according to claim 1; and
a medical connector comprising the female connector part to which the conversion adapter is attachable.

11. An assembly comprising:
the conversion adapter according to claim 1; and
the female connector part to which the conversion adapter is attached.

12. A conversion adapter attachable to a female connector part comprising a male screw part to which a luer lock-type first male connector part conforming to ISO 80369 is connectable, the conversion adapter comprising:
a cylindrical adapter body comprising:
a female screw part formed on an inner surface of the adapter body and screwable to the male screw part of the female connector part, and
a locking unit configured to lock to a second male connector part not conforming to ISO 80369, the locking unit being formed on an outer surface of the adapter body and comprising a recess engageable with a locking claw of the second male connector part.

13. A method of attaching a female connector to a male connector via an adaptor, the method comprising:
providing the female connector, which comprises a female connector part comprising a male screw part to which a luer lock-type first male connector part conforming to ISO 80369 is connectable;
providing the male connector, which comprises a male connector part not conforming to ISO 80639;
providing an adapter comprising:
a female screw part screwable to the male screw part of the female connector part; and
a locking unit; and
connecting the female connector to the male connector via the adaptor by:
attaching the female screw part of the adaptor to the male screw part of the female connector, and
attaching the locking unit of the adaptor to the male connector part of the male connector.
